(19) United States
(12) Patent Application Publication
(10) Pub. No.: US 2005/0215968 A1
(43) Pub. Date: Sep. 29, 2005

(54) Diaper construction

(76) Inventor: Francella Henderson, Inglewood, CA (US)

Correspondence Address: LAUSON & ASSOCIATES, 1334 PARK VIEW AVENUE, SUITE 100, MANHATTAN BEACH, CA, 90266, US

(21) Appl. No.: 10/811,383

(22) Filed: Mar. 29, 2004

(51) Int. Cl. A61F 13/15

(52) U.S. Cl. 604/385.14

(57) ABSTRACT

An throw-away diaper (10) insertable into a diaper wrap (12) that has an inner surface (24) with hook and loop connector parts (26,28, 30,32,34,36). Connector parts (38,40) can be mated with parts (34,36) producing releasable interconnection wrap sides. The diaper insert (14) is of geometry and dimensions similar to those of the wrap (12) and is preferably constructed of absorbent flushable paper.

### FIELD OF THE INVENTION

The present invention relates generally to a diaper construction, and, more particularly, to a combination of a throw-away diaper insert and an infant's diaper wrap.

### SUMMARY

The type of garment with which the insertable throw-away diaper to be described is most advantageously employed is what is termed a diaper wrap which includes a pants-like garment separable at both sides in order to enable laying out in a flat condition. Other garments that may be used for this purpose include training pants, pajamas or any other pant-like garment separable at the sides by buttons, snaps, zippers or other releasable fastening means enabling the garment to be opened to a relatively flat condition. When laid out flat the garment has an inside surface and an outside surface, and is of generally elongated rectangular shape with two centrally located inwardly curved side portions which adapt to the infant's legs when worn by the infant. At the four corner portions on the inner surface there are provided hook and loop connector parts spaced inwardly from the lateral edges. A second pair of hook and loop connector parts are secured to the inner surface of the garment just outwardly of the first described hook and loop connectors at one end of the garment. At the other end of the garment on the outer surface of the garment, there are provided further complementary hook and loop connector adjacent each lateral side of the garment for mating with the two opposite end internal connector parts of the garment.

The insert is of 'similar geometry to the garment and is constructed of multiple layers of an absorbent material which has the advantageous property of absorbing and not wetting the garment. Additionally, the diaper insert has an inner surface (i.e., surface to contact the infant) and an outer surface which on assembly contacts the inner surface of the garment. At the four corner portions of the outer surface of the insert there are provided individual hook and loop connector parts which have a generally L-shape with sides paralleling both the end edge and the lateral edge of the insert. With this L-shaped construction locking interconnection with the outer garment is adjustable over a range of positions to accommodate varying sizes of infants.

### DESCRIPTION OF THE DRAWING

In the accompanying drawing:

FIG. 1 is a perspective view of an infant's garment with a throw away diaper insert in accordance with the present invention;

FIG. 2 is an elevational view of the garment of FIG. 1 showing it partially released;

FIG. 3 shows a front view of an infant's garment modified for practicing the invention;

FIG. 4 shows the modified infant's garment of FIG. 3 with the insert in place;

FIG. 5 is a view similar to FIG. 4 showing part of the insert turned back and disconnected from the infant's garment; and

FIG. 6 shows the infant's garment and insert in separated spaced apart relationship.

### DESCRIPTION OF A PREFERRED EMBODIMENT

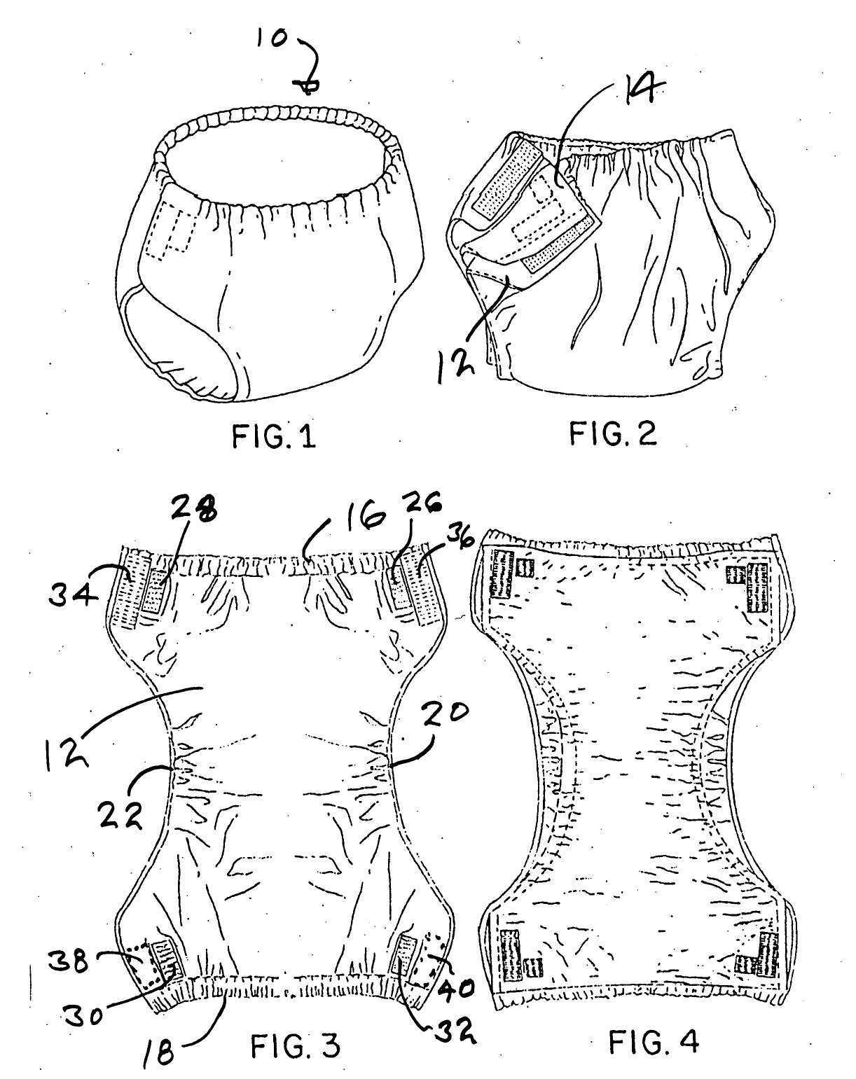

Turning now to the drawings and particularly FIGS. 1 and 2, the diaper arrangement of the present invention enumerated generally as 10 is seen to include in its major parts an outer pants-like garment 12 preferably what is termed a diaper wrap, which will be hereinafter referred to as a garment, within which a disposable throw-away, diaper insert 14 is unitarily and releasably secured for acting as diaper absorbing liquids while preventing substantial wetting of the garment. Also, as will be described, the combination garment and diaper insert can be quickly placed on an infant and just as quickly removed, while the throw away insert can be removed from the garment separately for disposal.

As can be seen best in FIG. 3, the garment 12 which is preferably what is sometimes termed a diaper wrap or training wrap, is typically constructed of a soft-to-the-touch textile fabric, such as cotton. The garment is generally rectangular and elongated with the two end edges 16 and 18 provided with an elastic so that they are shortened from the maximum width. In addition, the two longitudinal sides are curved inwardly as at 20 and 22 in order to accommodate the legs of the infant and aid in containing the insert.

What is to be the inner surface of the garment in use and identified as 24 in FIG. 3, includes first and second hook and loop connector parts 26 and 28, respectively, adjacent the edge 16 and a lateral edge. Similarly, third and fourth hook and loop connector parts 30 and 32 are located adjacent the opposite edge 18 and respectively adjacent the lateral peripheral edges. Each of the connector parts 26-32 are of the same character (e.g., loop connector parts). Just laterally outward from each of the two connector parts 26 and 28 there are located fifth and sixth connector parts 34 and 36, respectively.

On the outside surface of the garment and respectively closely adjacent the garment end edge 18, there are further connector parts 38 and 40 of such character as to enable mating with connector part 34 and 36 (i.e., loop connector).

It is instructive to note at this time that when the garment is folded from the position shown in FIG. 3 to the use position of FIGS. 1 and 2, that releasable interconnection of the garment sides is achieved by the connector parts 38 and 40 mating with connector parts 34 and 36.

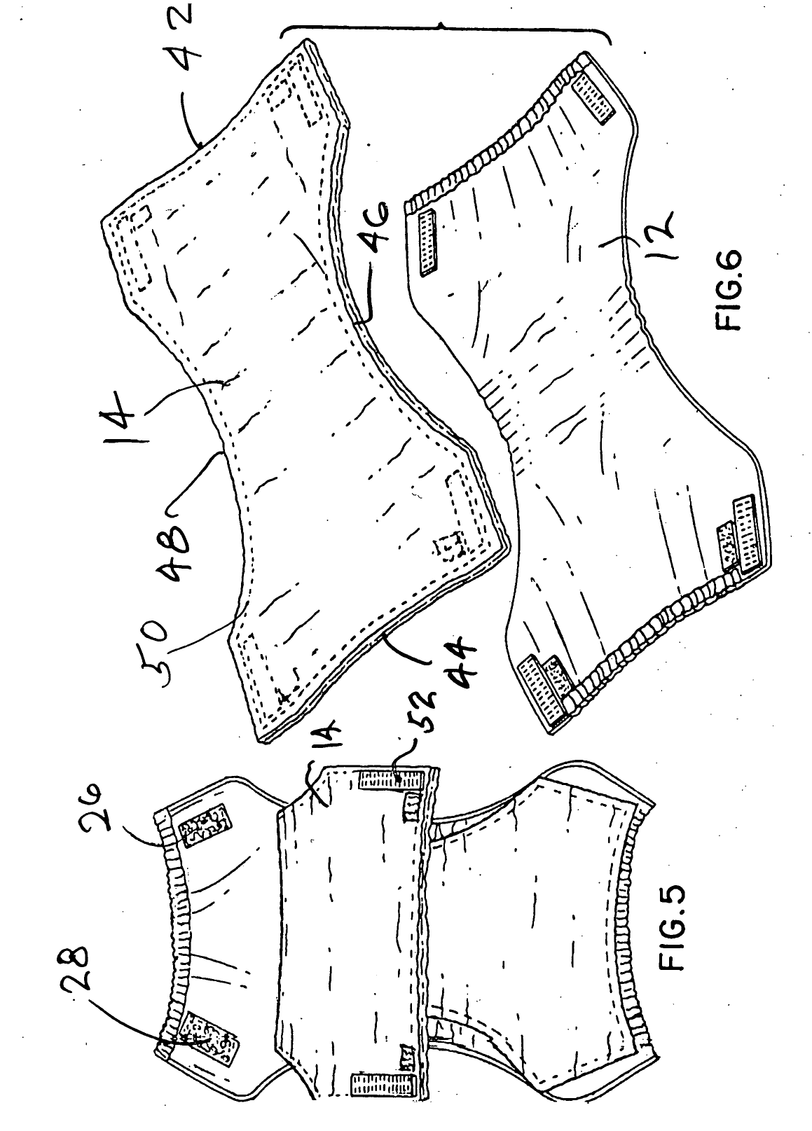

Returning now particularly to FIGS. 5 and 6, the throw-away diaper insert 14 is seen to include an elongated member of geometry and dimensions substantially similar to those of the garment. That is, the insert is generally rectangular with two narrow opposite end edges 42 and 44 with the long side edges being curved inwardly toward each other as at 46 and 48. Preferably the insert 14 is constructed of multiple layers of an absorbent flushable paper which are unitarily secured together along the peripheral edges by a line of stitching 50, for example. Alternatively, the paper layers composing the diaper insert may be secured at their edges by an adhesive.

Adjacent each corner of the insert, there is provided a generally L-shaped hook and loop connector part 52, its parts extending generally parallel to both the end edge and adjacent lateral edge. The connector parts 52 are all complementary to the connector parts 26-32 mounted on the garment 12.

In assembly the garment 12 is laid out flat as in FIG. 6 with the inside surface 24 facing upward. The diaper insert 14 is then laid down onto the garment with insert connector parts 52 being releasably secured to the garment connector parts 26-32. The combination garment and diaper insert may now be placed on the infant and the garment connector parts 38, 40 connected to connector parts 34, 36 holding the assembly on the infant.

The connector referred to herein as a hook and loop connector is well known and sold under the trade designation Velcro.

Although the invention has been described in connection with a preferred embodiment, it is understood that those skilled in the appertaining arts may make changes that come within the spirit of invention as described and within the ambit of the appended claims.

1. A diaper construction, comprising:
a diaper wrap garment constructed of a textile fabric having inner and outer surfaces;
a first plurality of hook and loop connector parts secured to the inner surface of the diaper wrap garment in a predetermined configuration;
a further pair of hook and loop connector parts secured to the outer surface of the diaper wrap and joinable with certain of the plurality of connectors;
a liquid absorbent biodegradable insert of generally sheetlike construction, said insert having a geometry closely similar to that of the diaper wrap garment; and
a second plurality of hook and loop connectors secured to the insert joinable with certain others of the first plurality of connectors for releasably securing the insert to the diaper wrap garment.

2. A diaper construction as in claim 1, in which the insert is constructed of a plurality of liquid absorbent paper sheets.

3. A diaper construction as in claim 1, in which the garment and insert are each elongated with generally parallel opposite end edges and opposite side edges curved inwardly toward each other.

4. A diaper construction as in claim 3, in which the second plurality of connectors include a first spaced apart pair arranged along one edge and a second spaced apart pair arranged along the opposite end edge, said second plurality of connectors each being of L-shape.

5. A diaper construction as in claim 4, in which the diaper wrap garment is pants-like and a cotton material.
